(19) United States
(12) Patent Application Publication
(10) Pub. No.: US 2017/0252008 A1
(43) Pub. Date: Sep. 7, 2017

(54) ULTRASONIC DEVICE AND ULTRASONIC PROBE

(71) Applicant: Seiko Epson Corporation, Tokyo (JP)

(72) Inventor: Robina ATSUCHI, Fujimi (JP)

(21) Appl. No.: 15/443,352

(22) Filed: Feb. 27, 2017

(30) Foreign Application Priority Data
Mar. 1, 2016 (JP) 2016-038646

(51) Int. Cl. A61B 8/00; G01S 7/52; A61B 8/14

(52) U.S. Cl. CPC A61B 8/4494; A61B 8/4444; A61B 8/54; A61B 8/145; G01S 7/52085

(57) ABSTRACT

An ultrasonic probe includes: an ultrasonic element group in which first ultrasonic element lines are arranged along a second direction crossing a first direction, a plurality of ultrasonic elements being arranged along the first direction in each of the first ultrasonic element lines; and a control unit for driving the ultrasonic element group. The control unit moves a focal point, which is a place through which ultrasonic waves emitted from the plurality of ultrasonic elements pass at the same time, along a virtual plane.

### BACKGROUND

1. Technical Field

The present invention relates to an ultrasonic device and an ultrasonic probe.

2. Related Art

An ultrasonic device is known in which a plurality of ultrasonic elements for emitting ultrasonic waves are arranged in a matrix on a substrate. In the ultrasonic device, a surface from which ultrasonic waves are emitted has a rectangular shape. The longitudinal direction of the rectangular shape is referred to as an azimuth direction, and a direction perpendicular to the azimuth direction is referred to as a slice direction.

JP-A-2006-61252 discloses an ultrasonic device for endoscopes. According to this, the position of a focal point is scanned by changing the timing, at which each ultrasonic element emits ultrasonic waves, in the azimuth direction. In the ultrasonic device, an acoustic lens is provided on a side from which ultrasonic waves are emitted.

In the slice direction, ultrasonic waves emitted from a plurality of ultrasonic elements overlap each other at the focal point due to the acoustic lens. A control unit for driving the ultrasonic elements moves the focal point of ultrasonic waves in the azimuth direction by driving the ultrasonic waves in a predetermined order. In addition, the focal point of ultrasonic waves is moved in a direction perpendicular to the substrate. Then, the ultrasonic device scans the focal point of ultrasonic waves in the azimuth direction and a direction perpendicular to the substrate. Then, an ultrasonic image is captured by receiving ultrasonic waves reflected at the focal point of ultrasonic waves.

In the ultrasonic device disclosed in the JP-A-2006-61252, an acoustic lens for condensing ultrasonic waves at the focal point is used. The ultrasonic device can obtain a sharp image by increasing the resolution at a place close to the focal point of the acoustic lens. However, as the distance from the focal point of the acoustic lens increases, the resolution decreases. Therefore, there has been a demand for an ultrasonic device capable of suppressing the occurrence of a place with low resolution.

### SUMMARY

An advantage of some aspects of the invention is to solve the problems described above, and the invention can be implemented as the following forms or application examples.

### APPLICATION EXAMPLE 1

An ultrasonic device according to this application example includes: an ultrasonic element group in which first ultrasonic element lines are arranged along a second direction crossing a first direction, a plurality of ultrasonic elements being arranged along the first direction in each of the first ultrasonic element lines; and a control unit that controls the ultrasonic element group. The control unit moves a focal point, which is a place through which ultrasonic waves emitted from the plurality of ultrasonic elements pass, along a virtual plane.

According to this application example, the ultrasonic device includes the ultrasonic element group and the control unit that controls the ultrasonic element group. In the ultrasonic element group, the first ultrasonic element lines each of which includes a plurality of ultrasonic elements arranged along the first direction are arranged along the second direction crossing the first direction. In the ultrasonic element group, each ultrasonic element emits an ultrasonic wave.

The control unit controls the timing at which each of the plurality of ultrasonic elements emits an ultrasonic wave. In addition, the control unit performs control so that ultrasonic waves emitted from the respective ultrasonic elements pass through a predetermined place at the same time. The predetermined place is referred to as a focal point. The control unit moves the focal point along the virtual plane. By detecting reflected waves, it is possible to detect the distribution of a place where the reflectance of the ultrasonic wave is high and a place where the reflectance of the ultrasonic wave is low on the virtual plane.

When an acoustic lens is used, ultrasonic waves pass through the predetermined place at the same time at a place close to the focal point of the acoustic lens. Therefore, the resolution can be increased. On the other hand, at a place far from the focal point of the acoustic lens, a place where the ultrasonic waves approach at the same time is away. Therefore, the resolution is reduced. Then, a high-resolution place and a low-resolution place are made. In the present embodiment, since the control unit moves the focal point along the virtual plane, the ultrasonic device does not require an acoustic lens for condensing ultrasonic waves at the predetermined place. Then, the control unit controls the ultrasonic elements so that ultrasonic waves emitted from the plurality of ultrasonic elements pass through the focal point. Therefore, the ultrasonic device can suppress the occurrence of a place with low resolution.

### APPLICATION EXAMPLE 2

In the ultrasonic device according to the application example, each of the ultrasonic elements may include a piezoelectric material interposed between first and second electrodes and an insulating layer interposed between the second electrode and a third electrode. An electrical resistor may be provided in the second electrode. The control unit may input a first pulse signal between the first electrode and the electrical resistor and input a second pulse signal between the first and third electrodes.

According to this application example, each ultrasonic element includes the piezoelectric material interposed between the first and second electrodes and the insulating layer interposed between the second and third electrodes. In addition, the electrical resistor is provided in the second electrode. The control unit inputs the first pulse signal between the first electrode and the electrical resistor, and inputs the second pulse signal between the first and third electrodes.

When the control unit inputs the first pulse signal to the electrical resistor, the first pulse signal is applied to the second electrode through the electrical resistor. Since the insulating layer is interposed between the second and third electrodes, the second electrode, the third electrode, and the insulating layer form the condenser. In addition, the control unit inputs the second pulse signal between the first and third electrodes.

The first and second pulse signals are signals switched between a reference voltage and a first voltage higher than the reference voltage. When the control unit sets the second pulse signal to have the reference voltage and sets the first pulse signal to have the first voltage, the first voltage is applied to the condenser. Then, the condenser holds the first voltage. Then, when the control unit sets the first and second pulse signals to have the first voltage, a voltage twice the first voltage is applied to the piezoelectric material. Then, since a current flows through the electrical resistor, the voltage applied to the piezoelectric material shifts to the first voltage. Therefore, it is possible to drive the piezoelectric material with a higher voltage than a voltage output from the control unit.

### APPLICATION EXAMPLE 3

In the ultrasonic device according to the application example, the control unit may drive the ultrasonic element at an end of the first ultrasonic element line with a voltage lower than a voltage for the ultrasonic element on a central side.

According to this application example, the control unit drives the ultrasonic element at the end of the first ultrasonic element line with a voltage lower than the voltage for the ultrasonic element on the central side. At this time, it is possible to reduce the amount of reflected waves from places other than the focal point, compared with a case in which the control unit drives the ultrasonic element at the end of the first ultrasonic element line with the same voltage as for the ultrasonic element on the central side.

### APPLICATION EXAMPLE 4

The ultrasonic device according to the application example may further include: a first wiring line that is connected to each ultrasonic element of the first ultrasonic element line to transmit the first pulse signal; and a second wiring line that is connected to the ultrasonic element of a second ultrasonic element line, which is arranged along the second direction, to transmit the second pulse signal.

According to this application example, each ultrasonic element of the first ultrasonic element line arranged along the first direction is connected through the first wiring line. The first pulse signal is input to the ultrasonic element through the first wiring line. In addition, each ultrasonic element of the second ultrasonic element line arranged along the second direction is connected through the second wiring line. The second pulse signal is input to the ultrasonic element through the second wiring line. Therefore, it is possible to reduce the number of wiring lines compared with a case where wiring lines for supplying the first and second pulse signals are provided for each ultrasonic element.

### APPLICATION EXAMPLE 5

An ultrasonic probe according to this application example includes: an ultrasonic element group in which first ultrasonic element lines are arranged along a second direction crossing a first direction, a plurality of ultrasonic elements being arranged along the first direction in each of the first ultrasonic element lines; and a control unit that controls the ultrasonic element group. The control unit moves a focal point, which is a place through which ultrasonic waves emitted from the plurality of ultrasonic elements pass, along a virtual plane.

According to this application example, the ultrasonic probe includes the ultrasonic element group and the control unit that controls the ultrasonic element group. In the ultrasonic element group, the first ultrasonic element lines each of which includes a plurality of ultrasonic elements arranged along the first direction are arranged along the second direction crossing the first direction. In the ultrasonic element group, each ultrasonic element emits an ultrasonic wave.

The control unit controls the timing at which each of the plurality of ultrasonic elements emits an ultrasonic wave. In addition, the control unit performs control so that ultrasonic waves emitted from the respective ultrasonic elements pass through a predetermined place at the same time. The predetermined place is referred to as a focal point. The control unit moves the focal point along the virtual plane. By detecting reflected waves, it is possible to detect the distribution of a place where the reflectance of the ultrasonic wave is high and a place where the reflectance of the ultrasonic wave is low on the virtual plane.

When an acoustic lens is used, ultrasonic waves pass through the predetermined place at the same time at a place close to the focal point of the acoustic lens. Therefore, the resolution can be increased. On the other hand, at a place far from the focal point of the acoustic lens, a place where the ultrasonic waves approach at the same time is away. Therefore, the resolution is reduced. Then, a high-resolution place and a low-resolution place are made. In the present embodiment, since the control unit moves the focal point along the virtual plane, the ultrasonic probe does not require an acoustic lens for condensing ultrasonic waves at the predetermined place. Then, the control unit controls the ultrasonic elements so that ultrasonic waves emitted from the plurality of ultrasonic elements pass through the focal point. Therefore, the ultrasonic probe can suppress the occurrence of a place with low resolution.

### BRIEF DESCRIPTION OF THE DRAWINGS

The invention will be described with reference to the accompanying drawings, wherein like numbers reference like elements.

FIG. 1 is a schematic perspective view showing the configuration of an ultrasonic image diagnostic apparatus according to a first embodiment.

FIG. 2 is a schematic perspective view showing the structure of an ultrasonic sensor.

FIG. 3 is a schematic diagram illustrating the focal point of ultrasonic waves.

FIG. 4 is a schematic diagram illustrating the focal point of ultrasonic waves.

FIG. 5 is a schematic diagram illustrating the trajectory of the focal point.

FIG. 6 is a schematic side sectional view showing the structure of an ultrasonic element.

FIG. 7 is a schematic side sectional view showing the structure of an ultrasonic element.

FIG. 8 is a circuit diagram of a piezoelectric element and a condenser.

FIG. 9 is a time chart illustrating a driving signal.

FIG. 10 is a time chart of a pulse signal supplied to second and third electrode wiring lines.

FIG. 11 is a graph illustrating a voltage applied to a piezoelectric element.

FIG. 12 is a schematic diagram illustrating the strength of an ultrasonic wave emitted from an ultrasonic sensor.

FIG. 13 is a circuit diagram of a piezoelectric element and a condenser according to a second embodiment.

FIG. 14 is a time chart illustrating a driving signal.

### DESCRIPTION OF EXEMPLARY EMBODIMENTS

Hereinafter, embodiments will be described with reference to the accompanying diagram. In each diagram, the scale of each member is adjusted in order to have a recognizable size.

### First Embodiment

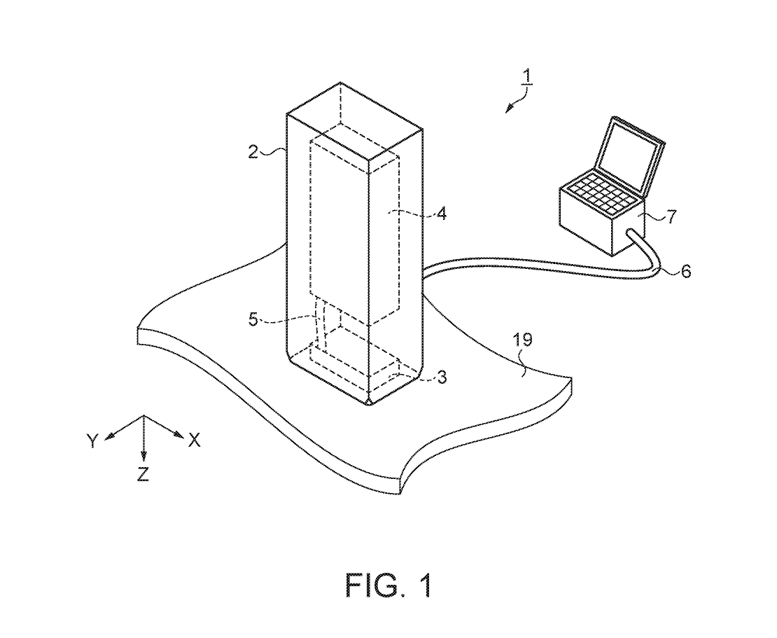

In the present embodiment, a characteristic example of an ultrasonic image diagnostic apparatus including an ultrasonic probe will be described with reference to the accompanying diagrams. An ultrasonic image diagnostic apparatus according to a first embodiment will be described with reference to FIGS. 1 to 12. FIG. 1 is a schematic perspective view showing the configuration of an ultrasonic image diagnostic apparatus. As shown in FIG. 1, an ultrasonic image diagnostic apparatus 1 includes an ultrasonic probe 2 as an ultrasonic device. The ultrasonic probe 2 has an approximately rectangular parallelepiped shape which is long in one direction. The longitudinal direction of the ultrasonic probe 2 is defined as a Z direction. The surface of the ultrasonic probe 2 in the +Z direction is an approximately flat surface, and the planar shape is a rectangle. Directions in which two sides of the planar shape perpendicular to each other extend are defined as an X direction and a Y direction.

An ultrasonic sensor 3 is provided on the +Z direction side of the ultrasonic probe 2. On the surface of the ultrasonic probe 2 on the +Z direction side, the ultrasonic sensor 3 is exposed from the housing. A control unit 4 for controlling the ultrasonic sensor 3 is provided in the ultrasonic probe 2, and the ultrasonic sensor 3 and the control unit 4 are connected to each other by a cable 5. A central processing unit (CPU) and a storage device are provided in the control unit 4. Data of driving waveforms for driving the ultrasonic sensor 3 or a program indicating a procedure for driving the ultrasonic sensor 3 is stored in the storage device. Then, the CPU drives the ultrasonic sensor 3 by outputting driving waveforms to the ultrasonic sensor 3 along the program.

The ultrasonic probe 2 is connected to a control device 7 through a cable 6. The control device 7 is a device to which a data signal output from the ultrasonic probe 2 is input and which analyzes and displays the data signal.

The ultrasonic probe 2 is used in a state of being pressed against the surface of a living body 19. The ultrasonic probe 2 emits ultrasonic waves toward the living body 19 from the ultrasonic sensor 3. Then, the ultrasonic sensor 3 receives reflected waves that are reflected from the inside of the living body 19. Since the time taken for reflection and return of the reflected wave differs depending on the reflected surface, it is possible to examine the internal structure of the living body 19 in a non-destructive manner by analyzing the return time of the reflected wave. The signal of the reflected wave received by the ultrasonic sensor 3 is output to the control unit 4. The control unit 4 includes an analog-to-digital (A/D) conversion section, and converts the signal of the reflected wave into digital data. Then, a data signal obtained by conversion into the digital data is transmitted to the control device 7 through the cable 5, the control unit 4, and a cable 6. The control device 7 receives and analyzes the data signal of the reflected wave. Then, the control device 7 converts the internal structure of the living body 19 into an image, and displays the image.

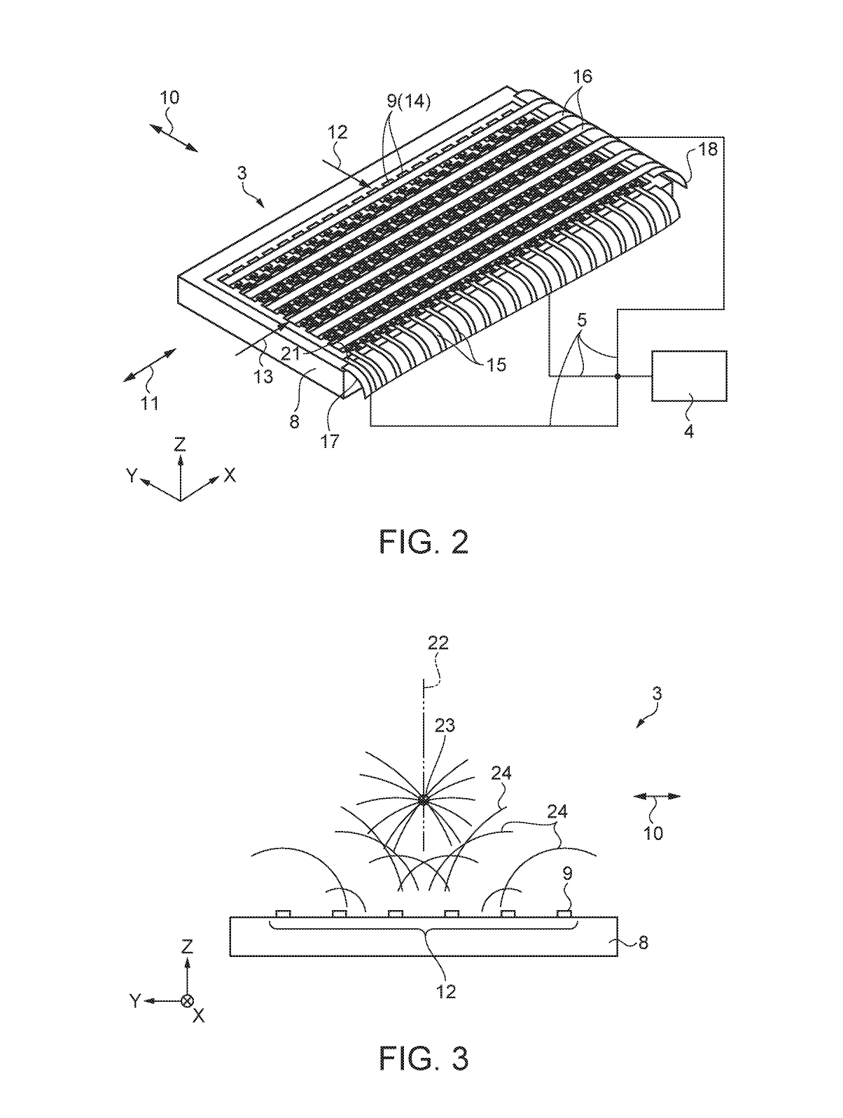

FIG. 2 is a schematic perspective view showing the structure of an ultrasonic sensor. As shown in FIG. 2, the ultrasonic sensor 3 includes a substrate 8. Ultrasonic elements 9 are provided in a matrix on the surface of the substrate 8 on the +Z direction side. The Y direction in FIG. 2 is defined as a first direction 10, and the X direction is defined as a second direction 11. The ultrasonic elements 9 are aligned in the first and second directions 10 and 11. The ultrasonic elements 9 arranged in the first direction 10 are defined as a first ultrasonic element line 12, and the ultrasonic elements 9 arranged in the second direction 11 are defined as a second ultrasonic element line 13. The ultrasonic element 9 has a function of emitting ultrasonic waves and a function of receiving reflected waves and converting the reflected waves into an electrical signal. In addition, the ultrasonic sensor 3 includes an element for emitting ultrasonic waves and an element for receiving reflected waves and converting the reflected waves into an electrical signal separately from each other.

In the present embodiment, for example, for easy understanding of explanation, the number of first ultrasonic element lines 12 in FIG. 2 is set to 20, and the number of second ultrasonic element lines 13 is set to 6. Accordingly, in the ultrasonic sensor 3, an ultrasonic element group 14 including 120 ultrasonic elements 9 on the substrate 8 is provided. If the number of first ultrasonic element lines 12 is 32 or more and the number of second ultrasonic element lines 13 is 256 or more, the resolution is increased. In this case, it is easy to see the detected ultrasonic image.

Each ultrasonic element 9 of the first ultrasonic element line 12 is connected to a second electrode wiring line 15 as a first wiring line extending in the first direction 10. Similarly, each ultrasonic element 9 of the second ultrasonic element line 13 is connected to a third electrode wiring line 16 as a second wiring line extending in the second direction 11. A flexible cable 17 is provided at the end of the substrate 8 on the −Y direction side, and the second electrode wiring line 15 is connected to the flexible cable 17. A flexible cable 18 is provided at the end of the substrate 8 on the +X direction side, and the third electrode wiring line 16 is connected to the flexible cable 18. Accordingly, it is possible to reduce the number of wiring lines compared with a case of supplying the driving signal to each ultrasonic element 9.

Each ultrasonic element 9 is connected to a first electrode wiring line 21, and the first electrode wiring line 21 is also connected to the flexible cable 17. The second and third electrode wiring lines 15 and 16 are connected to the control unit 4 through the cable 5. Then, the control unit 4 outputs a voltage signal to each ultrasonic element 9 through the second electrode wiring line 15, the third electrode wiring line 16, and the first electrode wiring line 21. The ultrasonic element 9 receives the voltage signal, and emits ultrasonic waves according to the voltage signal.

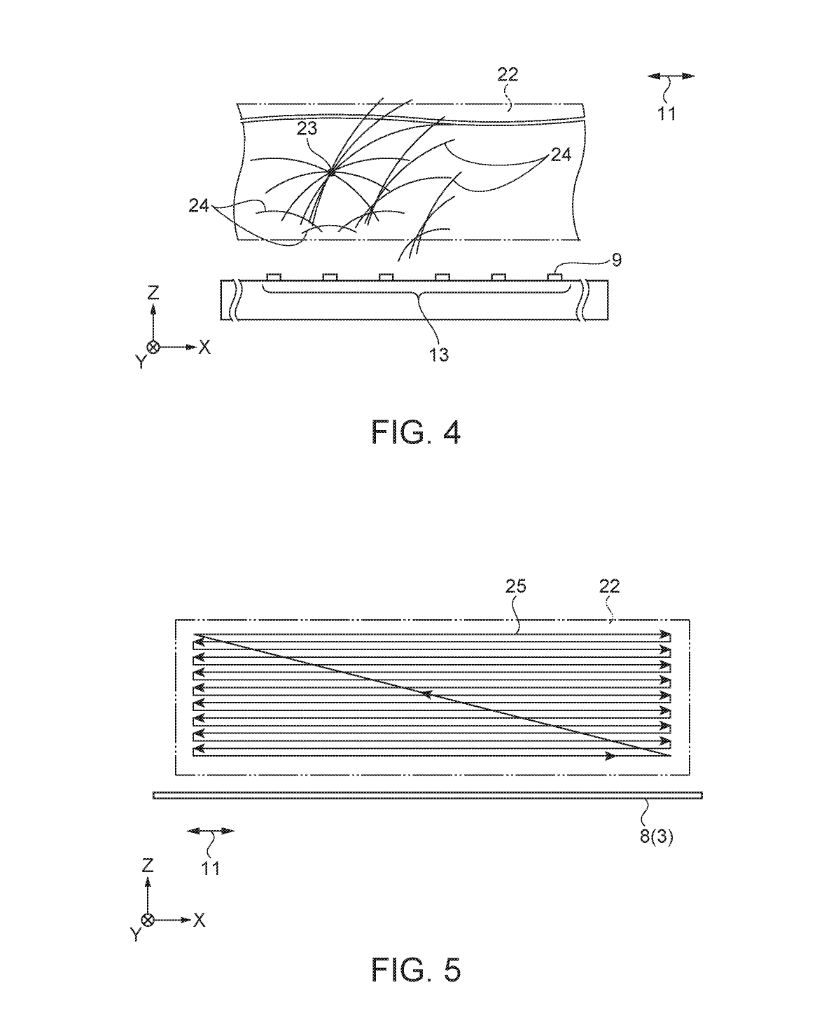

FIGS. 3 and 4 are schematic diagrams illustrating the focal point of ultrasonic waves. FIG. 3 shows the first ultrasonic element line 12, and FIG. 4 shows a part of the second ultrasonic element line 13. As shown in FIG. 3, the control unit 4 assumes a virtual plane 22 on the +Z direction side of the ultrasonic sensor 3. The virtual plane 22 is a plane extending in the X and Z directions. The position of the virtual plane 22 in the Y direction is not particularly limited. In the present embodiment, for example, the virtual plane 22 is set at the center of the six ultrasonic elements 9 forming the first ultrasonic element line 12. Then, the control unit 4 assumes a focal point 23 on the virtual plane 22. The control unit 4 causes the ultrasonic elements 9 to emit an ultrasonic wave 24 in order from the ultrasonic element 9 located at a place far from the focal point 23 to the ultrasonic element 9 located at a place close to the focal point 23.

In addition, the control unit 4 controls the timing, at which each ultrasonic element 9 emits the ultrasonic wave 24, so that the ultrasonic waves 24 emitted from the respective ultrasonic elements 9 of the first ultrasonic element line 12 pass through the focal point 23 at the same time. First, the control unit 4 calculates the distance between the focal point 23 and each ultrasonic element 9. Then, the movement time until the ultrasonic wave 24 reaches the focal point 23 is calculated by dividing the calculated distance by the speed of the ultrasonic wave 24. Then, the ultrasonic wave 24 is emitted from each ultrasonic element 9 while shifting the timing corresponding to the difference from the movement time. Accordingly, the first ultrasonic element line 12 can make the ultrasonic waves 24 pass through the focal point 23 at the same time. In addition, the control unit 4 may store the result calculated once in a storage device. In addition, the CPU may receive an operation result from the storage device and output a driving signal to the ultrasonic element group 14.

As shown in FIG. 4, the control unit 4 assumes the focal point 23 on the virtual plane 22. Also in the second ultrasonic element line 13, the control unit 4 causes the ultrasonic elements 9 to emit the ultrasonic wave 24 in order from the ultrasonic element 9 located at a place far from the focal point 23 to the ultrasonic element 9 located at a place close to the focal point 23. In addition, the control unit 4 controls the timing, at which each ultrasonic element 9 emits the ultrasonic wave 24, so that the ultrasonic waves 24 emitted from the respective ultrasonic elements 9 of the second ultrasonic element line 13 pass through the focal point 23 at the same time. The control unit 4 emits the ultrasonic wave 24 from each ultrasonic element 9 of the second ultrasonic element line 13 using the same method as the method performed for the first ultrasonic element line 12. Accordingly, the second ultrasonic element line 13 can make the ultrasonic waves 24 pass through the focal point 23 at the same time.

The control unit 4 controls the emission timing of the ultrasonic wave 24 from the ultrasonic element 9 of the first ultrasonic element line 12 and the emission timing of the ultrasonic wave 24 from the second ultrasonic element line 13 at the same time. Therefore, the ultrasonic waves 24 emitted from many ultrasonic elements 9 of the ultrasonic sensor 3 pass through the focal point 23 at the same time. At this time, since the sound pressure of the ultrasonic wave 24 is increased at the focal point 23, strong reflected waves can be generated when there is a member for reflecting the ultrasonic wave 24 at the focal point 23.

FIG. 5 is a schematic diagram illustrating the trajectory of the focal point. The control unit 4 moves the focal point 23 along the virtual plane 22. A trajectory 25 shown in FIG. 5 is a movement trajectory of the focal point 23 along the virtual plane 22. As indicated by the trajectory 25, the focal point 23 is scanned in the second direction 11 and the Z direction. The second direction 11 is a main scanning direction, and the Z direction is a sub-scanning direction.

When an acoustic lens is used, the ultrasonic waves 24 pass through a predetermined place at the same time at a place close to the focal point of the acoustic lens. In this case, therefore, the resolution can be increased. On the other hand, at a place far from the focal point of the acoustic lens, a place where the ultrasonic waves approach at the same time is away. In this case, therefore, the resolution is reduced. Then, a high-resolution place and a low-resolution place are made. In the present embodiment, since the control unit 4 moves the focal point along the virtual plane 22, the ultrasonic probe does not require an acoustic lens for condensing the ultrasonic waves 24 at a predetermined place. Then, the control unit 4 controls the ultrasonic elements 9 so that the ultrasonic waves 24 emitted from the plurality of ultrasonic elements 9 pass through the focal point. Therefore, the ultrasonic probe 2 can suppress the occurrence of a place with low resolution.

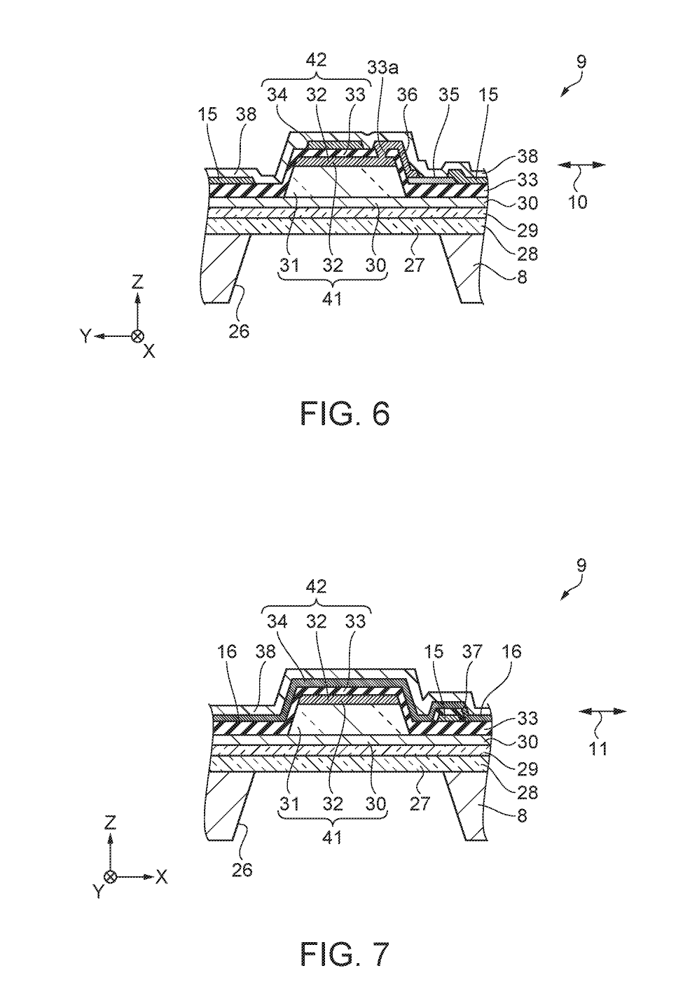

FIGS. 6 and 7 are schematic side sectional views showing the structure of an ultrasonic element. As shown in FIGS. 6 and 7, a recessed portion 26 is provided at a place facing the ultrasonic element 9 on the substrate 8. The thickness of apart of the substrate 8 is reduced by the recessed portion 26, and a place where the thickness is small is a vibrating portion 27. The substrate 8 is a silicon substrate, and the recessed portion 26 is formed by etching. In the vibrating portion 27, a silicon oxide film 28 and a zirconium oxide film 29 are laminated.

A first electrode 30 is provided on the +Z direction side of the vibrating portion 27. The first electrode 30 is connected to the first electrode wiring line 21. The first electrode 30 is formed by forming a metal layer and patterning the metal layer using a photolithography method.

A piezoelectric material 31 is provided on the first electrode 30. The piezoelectric material 31 is formed by forming a pyroelectric material layer, which is a layer of the material of the piezoelectric material 31, and patterning the pyroelectric material layer using a photolithography method. The pyroelectric material layer is a layer of a lead zirconate titanate (PZT) film.

The pyroelectric material layer is provided using a sputtering method or a sol-gel method. In the sputtering method, using a PZT sintered body of a specific component as a target for sputtering, an amorphous piezoelectric film precursor layer is formed on the substrate 8 by sputtering. Then, the amorphous piezoelectric film precursor layer is heated, crystallized, and sintered.

In the sol-gel method, a sol that is a hydrate complex of hydroxide, such as titanium, zirconium, and lead that are materials of the pyroelectric material layer, is generated. A gel is obtained by dehydrating the sol. The gel is heated and baked to generate a pyroelectric material layer that is an inorganic oxide.

A second electrode 32 is provided on the piezoelectric material 31. The second electrode 32 is formed by forming a metal layer and patterning the metal layer using a photolithography method. An insulating layer 33 is provided on the second electrode 32. In the insulating layer 33, a through hole 33a is formed on the second electrode 32. The insulating layer 33 is provided so as to cover the piezoelectric material 31 and the first electrode 30. The insulating layer 33 is also provided at a place where the zirconium oxide film 29 is exposed. The insulating layer 33 is formed by forming a layer using a vacuum deposition method or the like and patterning the layer using a photolithography method.

A third electrode 34 is provided at a place facing the second electrode 32 on the insulating layer 33. The third electrode 34 is connected to the third electrode wiring line 16 extending in the second direction 11. More specifically, a metal layer at the place facing the second electrode 32 is the third electrode 34, and a metal film at a place not facing the second electrode 32 is the third electrode wiring line 16.

On the insulating layer 33, a resistor 35 as an electrical resistor is provided on the −Y direction side of the piezoelectric material 31. The resistor 35 is a film having electrical resistance. Although the material of the resistor 35 is not particularly limited, for example, carbon is used as a main material of the resistor 35, in the present embodiment. The resistor 35 is formed by forming a layer containing carbon as a main material and patterning the layer using a photolithography method.

A connection wiring line 36 is provided between one end of the resistor 35 and the second electrode 32 in order to connect the resistor 35 and the second electrode 32 to each other. The connection wiring line 36 is provided in the through hole 33a, and is connected to the second electrode 32 through the through hole 33a. The other end of the resistor 35 is connected to the second electrode wiring line 15.

On the insulating layer 33, the second electrode wiring line 15 is provided passing through the +X direction side of the piezoelectric material 31. The second electrode wiring line 15 is a wiring line extending in the first direction 10. An insulating layer 37 is provided so as to cover a part of the second electrode wiring line 15. The insulating layer 37 is formed by forming a layer using a vacuum deposition method or the like and patterning the layer using a photolithography method.

Then, the third electrode wiring line 16 is provided on the insulating layer 37. The third electrode wiring line 16 is provided in the same step as for the third electrode 34. Therefore, the resistor 35, the second electrode wiring line 15, the connection wiring line 36, and the insulating layer 37 are provided in steps before the third electrode 34 is provided.

Materials of the first electrode 30, the second electrode 32, and the third electrode 34 are not particularly limited. In the present embodiment, for example, iridium oxide and platinum are used, and a platinum film is provided on an iridium oxide film. In addition, an insulating layer 38 is provided so as to cover the second electrode wiring line 15, the third electrode wiring line 16, and the third electrode 34, thereby preventing electric leakage between wiring lines. Materials of the insulating layer 33, the insulating layer 37, and the insulating layer 38 are not particularly limited. In the present embodiment, for example, silicon oxide or alumina oxide can be used.

A piezoelectric element 41 is configured by interposing the piezoelectric material 31 between the first electrode 30 and the second electrode 32. In addition, a condenser 42 is configured by interposing the insulating layer 33 between the second electrode 32 and the third electrode 34.

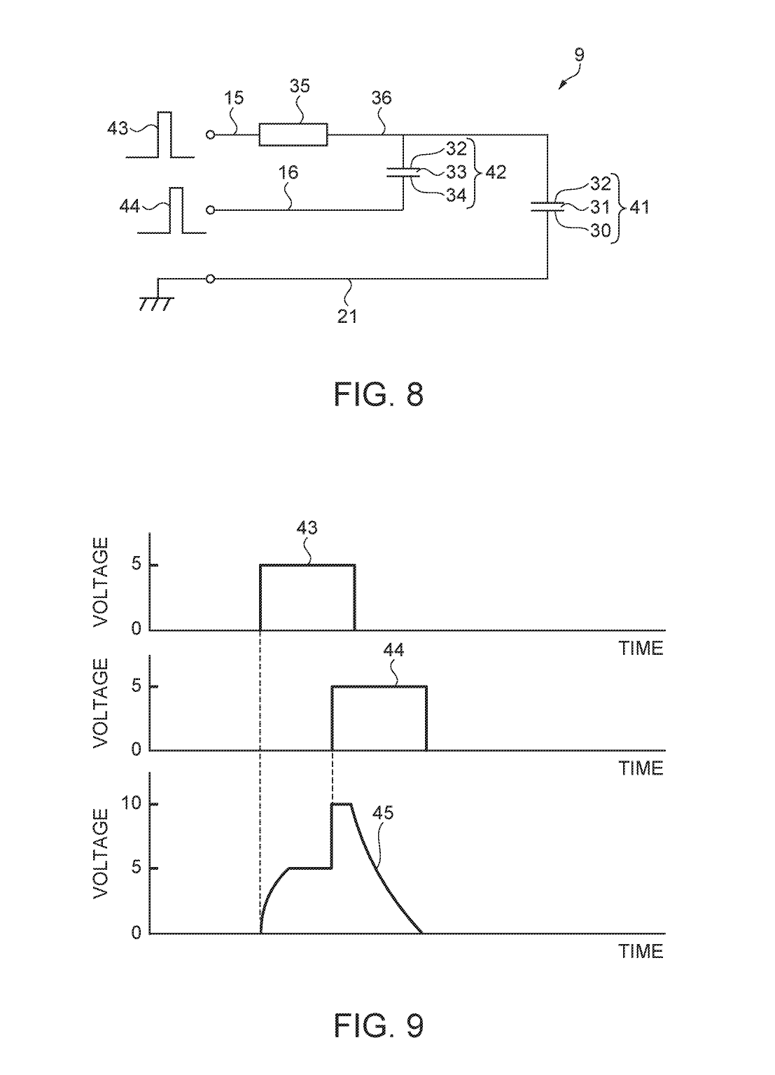

FIG. 8 is a circuit diagram of a piezoelectric element and a condenser. As shown in FIG. 8, the first electrode 30 of the piezoelectric element 41 is connected to the first electrode wiring line 21. The first electrode wiring line 21 is grounded. The second electrode 32 of the piezoelectric element 41, the second electrode 32 of the condenser 42, and the end of the resistor 35 are connected to each other by the connection wiring line 36. The second electrode wiring line 15 is connected to the other end of the resistor 35, and a first pulse signal 43 is supplied from the second electrode wiring line 15. The third electrode wiring line 16 is connected to the third electrode 34 of the condenser 42, and a second pulse signal 44 is supplied from the third electrode wiring line 16.

FIG. 9 is a time chart illustrating a driving signal. In FIG. 9, the vertical axis indicates a voltage, and the voltage on the upper side in the diagram is higher than that on the lower side. In addition, the horizontal axis indicates the transition of time, and the time transitions to the right side from the left side in the diagram. The first pulse signal 43 in the diagram is a voltage signal applied between the first electrode wiring line 21 and the second electrode wiring line 15. The second pulse signal 44 is a voltage signal applied between the first electrode wiring line 21 and the third electrode wiring line 16.

A driving waveform 45 is a waveform for driving the piezoelectric element 41, and indicates the transition of a voltage applied between the first electrode 30 and the second electrode 32. First, when the voltages of the first pulse signal 43 and the second pulse signal 44 are 0 V, the voltage of the driving waveform 45 is also 0 V.

Then, the first pulse signal 43 rises to 5 V. Then, a current flows through the resistor 35, so that electric charges are accumulated in the condenser 42. As a result, the driving waveform 45 rises to 5V. Then, the second pulse signal 44 rises to 5 V. Accordingly, the electric potential of the third electrode 34 becomes an electric potential of 5 V with respect to the first electrode 30. Since the condenser 42 is charged to 5 V, the electric potential of the second electrode 32 becomes an electric potential of 10 V with respect to the first electrode 30.

At this time, since a voltage of 10 V is applied to the piezoelectric element 41, the driving waveform 45 becomes 10 V. The piezoelectric element 41 is set so as not to emit the ultrasonic wave 24 when the applied voltage is 5 V and so as to emit the ultrasonic wave 24 when the applied voltage abruptly rises from 5 V to 10 V. Therefore, when the second pulse signal 44 rises from 0 V to 5 V, the piezoelectric element 41 emits the ultrasonic wave 24.

Since the electric potential of the second electrode wiring line 15 is 5 V, the charge of the second electrode 32 moves to the second electrode wiring line 15 through the resistor 35. In addition, since the first pulse signal 43 becomes 0 V, the charge of the second electrode 32 further moves to the second electrode wiring line 15 through the resistor 35. Accordingly, the voltage of the driving waveform 45 gradually drops to become 0 V. Then, the voltage of the second pulse signal 44 also drops to 0 V. Then, the piezoelectric material 31 returns to the initial state in which no voltage is applied. Therefore, the control unit 4 applies a 5 V signal to each of the first pulse signal 43 and the second pulse signal 44 so that the piezoelectric element 41 can be driven at 10 V.

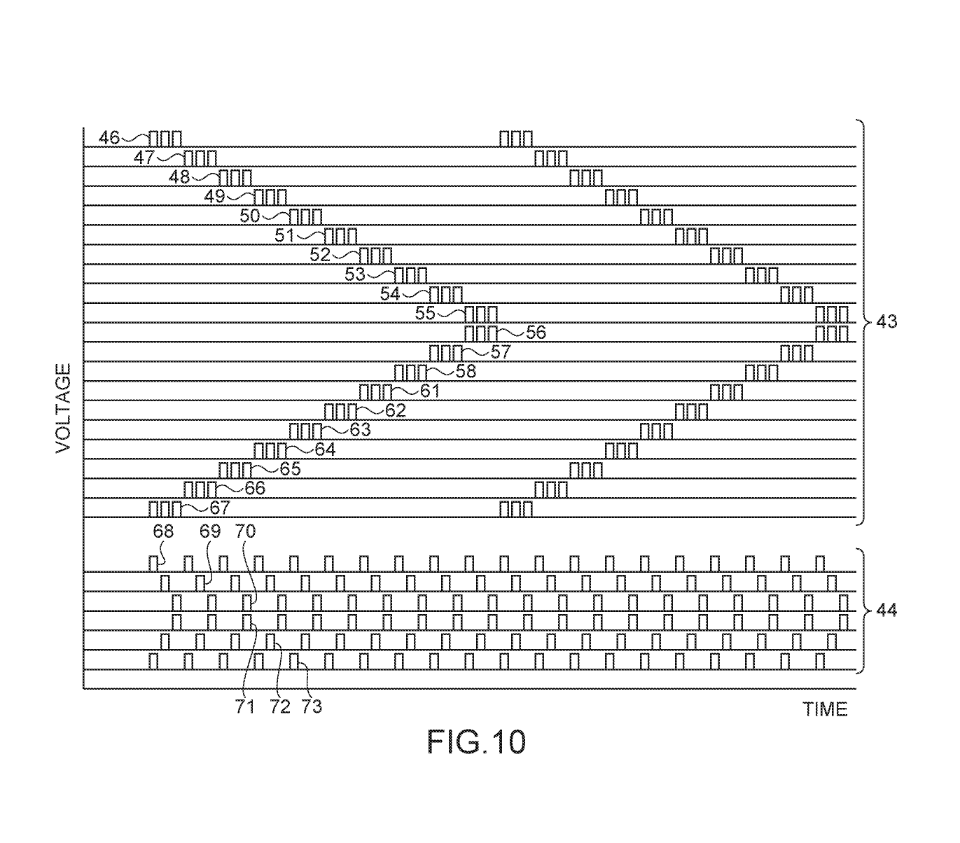

FIG. 10 is a time chart of the pulse signal supplied to the second and third electrode wiring lines. In FIG. 10, the vertical axis indicates a voltage, and the voltage on the upper side in the diagram is higher than that on the lower side. In addition, the horizontal axis indicates the transition of time, and the time transitions to the right side from the left side in the diagram. The upper stage in the diagram is the first pulse signal 43, and is a signal supplied to the second electrode wiring line 15.

A signal 46 is a signal applied to the second electrode wiring line 15 at the end on the −X direction side. A signal 47 is a signal applied to the second electrode wiring line 15 located at the second from the end on the −X direction side. Similarly, signals 48 to 67 are signals applied to the third to twentieth second electrode wiring lines 15 from the end on the −X direction side, respectively.

The lower stage in the diagram is the second pulse signal 44, and is a signal supplied to the third electrode wiring line 16. A signal 68 is a signal applied to the third electrode wiring line 16 at the end on the +Y direction side. A signal 69 is a signal applied to the third electrode wiring line 16 located at the second from the end on the +Y direction side. Similarly, signals 70 to 73 are signals applied to the third to sixth third electrode wiring lines 16 from the end on the +Y direction side, respectively.

Among the ultrasonic elements 9 arranged in a matrix, the ultrasonic element 9 that is an n-th ultrasonic element from the end on the −X direction side and an m-th ultrasonic element from the end on the +Y direction side is defined as an element (n, m). That is, an element (1, 1) is the ultrasonic element 9 at the end on the −X direction side and the end on the +Y direction side. An element (20, 6) is the ultrasonic element 9 at the end on the +X direction side and the end on the −Y direction side. In addition, a state in which each of the signals 46 to 73 is 0 V is set to “L”, and a state in which each of the signals 46 to 73 is 5 V is set to “H”.

First, the control unit 4 changes the signals 46 and 67 to “H” from “L”. In addition, the control unit 4 changes the signals 68 and 73 to “H” from “L”. Accordingly, the ultrasonic wave 24 is emitted from the element (1, 1), the element (1, 6), the element (20, 1), and the element (20, 6). Then, the control unit 4 changes the signals 46, 67, 68, and 73 to “L” from “H”.

Then, the control unit 4 changes the signals 46 and 67 to “H” from “L”. In addition, the control unit 4 changes the signals 69 and 72 to “H” from “L”. Accordingly, the ultrasonic wave 24 is emitted from the element (1, 2), the element (1, 5), the element (20, 2), and the element (20, 5). Then, the control unit 4 changes the signals 46, 67, 69, and 72 to “L” from “H”.

In addition, the control unit 4 changes the signals 46 and 67 to “H” from “L”. In addition, the control unit 4 changes the signals 70 and 71 to “H” from “L”. Accordingly, the ultrasonic wave 24 is emitted from the element (1, 3), the element (1, 4), the element (20, 3), and the element (20, 4). Then, the control unit 4 changes the signals 46, 67, 70, and 71 to “L” from “H”. As a result, in the first ultrasonic element line 12 at the end in the −X direction and the first ultrasonic element line 12 at the end in the +X direction, the ultrasonic waves 24 are emitted in order from the place far from the virtual plane 22 toward the place close to the virtual plane 22.

Then, the control unit 4 changes the signals 47, 66, 68, and 73 to “H” from “L” in the same procedure. Then, the control unit 4 changes each signal to “L” from “H”. As a result, the ultrasonic wave 24 is emitted from the element (2, 1), the element (2, 6), the element (19, 1), and the element (19, 6). Then, the ultrasonic wave 24 is emitted from the element (2, 2), the element (2, 5), the element (19, 2), and the element (19, 5). In addition, the ultrasonic wave 24 is emitted from the element (2, 3), the element (2, 4), the element (19, 3), and the element (19, 4). Thus, in the first ultrasonic element line 12 located at the second from the end in the −X direction and the first ultrasonic element line 12 located at the second from the end in the +X direction, the ultrasonic waves 24 are emitted in order from the place far from the virtual plane 22 toward the place close to the virtual plane 22.

Then, in the same procedure, the control unit 4 causes the ultrasonic waves 24 to be emitted in order from the place far from the virtual plane 22 toward the place close to the virtual plane 22 in the first ultrasonic element line 12 located at the third from the end in the −X direction and the first ultrasonic element line 12 located at the third from the end in the +X direction. In this manner, the control unit 4 causes the ultrasonic waves 24 to be emitted from the first ultrasonic element line 12 in order from the end side in the −X direction toward the central side. In parallel to this, the ultrasonic waves 24 are emitted in order from the end side in the +X direction toward the central side. Accordingly, the ultrasonic waves 24 can be made to pass through the focal point 23 at the same time at the center in the second direction 11.

Then, the control unit 4 changes the patterns of the first pulse signal 43 and the second pulse signal 44. Then, the control unit 4 changes the timing of the ultrasonic wave 24 emitted from each ultrasonic element 9. As a result, the ultrasonic sensor 3 can move the focal point 23 along the trajectory 25 of the virtual plane 22.

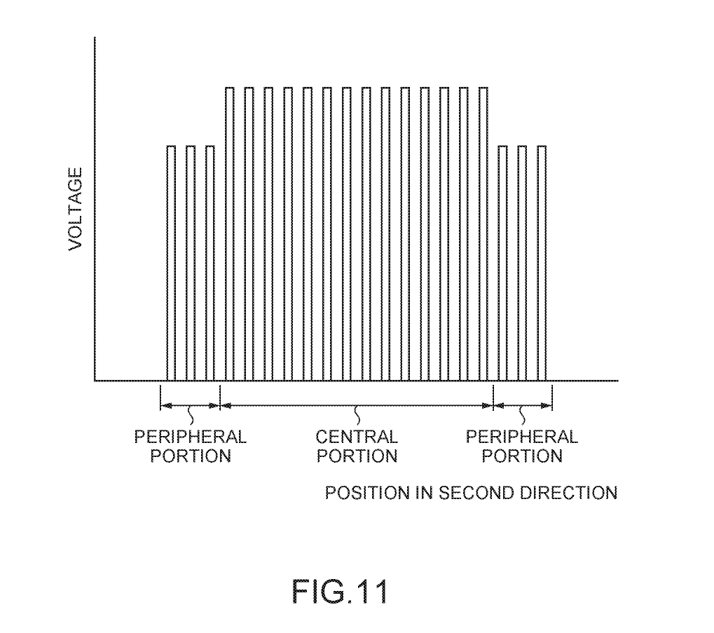

FIG. 11 is a graph illustrating a voltage applied to the piezoelectric element. In FIG. 11, the vertical axis indicates a voltage applied to the piezoelectric element 41, and the voltage on the upper side is higher than that on the lower side. The horizontal axis indicates a position in the second direction 11. A voltage applied to the piezoelectric element 41 in a peripheral portion in the second direction 11 is set to a voltage lower than a voltage applied to the central piezoelectric element 41. Therefore, for example, the first ultrasonic element lines 12 of three lines from the end in the −X direction and the first ultrasonic element lines 12 of three lines from the end in the +X direction are defined as the peripheral first ultrasonic element lines 12. The central first ultrasonic element lines 12 are from the first ultrasonic element lines 12 located at the fourth from the end in the −X direction to the first ultrasonic element lines 12 located at the fourth from the end in the +X direction. Then, for example, a voltage of 10 V is applied to the piezoelectric material 31 in the central first ultrasonic element line 12, and a voltage of 9 V is applied to the piezoelectric material 31 in the peripheral first ultrasonic element line 12.

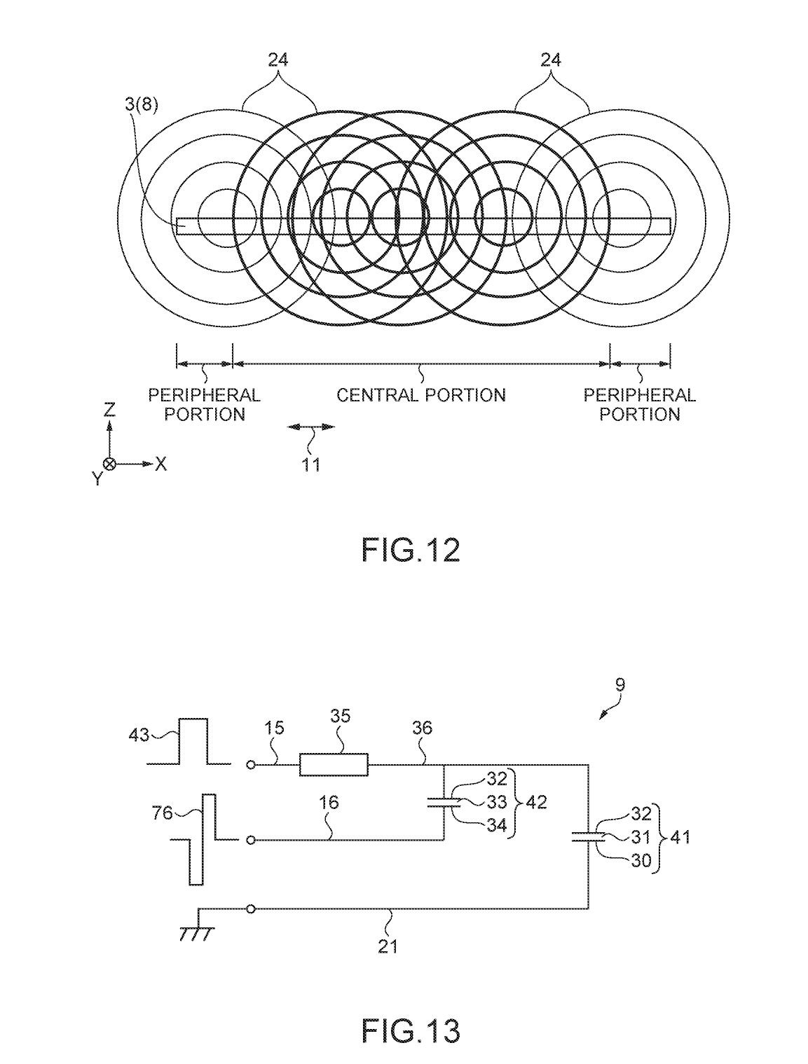

FIG. 12 is a schematic diagram illustrating the strength of the ultrasonic wave emitted from the ultrasonic sensor. As shown in FIG. 12, the ultrasonic wave 24 is emitted from the ultrasonic sensor 3. For the ultrasonic wave 24, the thickness of the line indicates the strength of the sound pressure. A thick line indicates the ultrasonic wave 24 with high sound pressure, and a thin line indicates the ultrasonic wave 24 with low sound pressure.

For the ultrasonic wave 24 emitted from the ultrasonic sensor 3, the sound pressure on the end side in the second direction 11 is lower than that on the central side. An ultrasonic image captured by the ultrasonic sensor 3 is an image of a place facing the ultrasonic sensor 3. A large proportion of the ultrasonic waves 24 emitted from the ultrasonic elements 9 at places close to the periphery of the ultrasonic sensor 3 is emitted to a part other than the place to be imaged. In the present embodiment, the sound pressure of the ultrasonic wave 24 emitted from the peripheral portion of the ultrasonic sensor 3 is set to be lower than that emitted from the central portion. Therefore, the ultrasonic probe 2 can reduce the amount of reflected waves from a place that is not imaged by emitting the ultrasonic wave 24 with high sound pressure to a place to be imaged. In other words, it is possible to reduce the amount of reflected waves from places other than the movement range of the focal point 23. As a result, it is possible to make a captured ultrasonic image clear.

Also in the first direction 10, the control unit 4 sets the sound pressure of the ultrasonic wave 24 emitted from the end side of the ultrasonic sensor 3 to be lower than that of the ultrasonic wave 24 emitted from the central side of the ultrasonic sensor 3. Also at this time, it is possible to reduce the amount of reflected waves from places other than the movement range of the focal point 23. As a result, it is possible to make a captured ultrasonic image clear.

As described above, according to the present embodiment, the following effect is obtained.

(1) According to the present embodiment, the ultrasonic probe 2 includes the ultrasonic element group 14 and the control unit 4 for driving the ultrasonic element group 14. In the ultrasonic element group 14, the first ultrasonic element line 12 in which a plurality of ultrasonic elements 9 are arranged along the first direction 10 is arranged along the second direction 11 perpendicular to the first direction 10. In the ultrasonic element group 14, each ultrasonic element 9 emits the ultrasonic wave 24.

The control unit 4 controls the timing at which each of the plurality of ultrasonic elements 9 emits the ultrasonic wave 24. In addition, the control unit 4 performs control so that the ultrasonic waves 24 emitted from the respective ultrasonic elements 9 pass through the focal point 23 at the same time. The control unit 4 moves the focal point 23 along the virtual plane 22. By detecting reflected waves, it is possible to detect the distribution of a place where the reflectance of the ultrasonic wave 24 is high and a place where the reflectance of the ultrasonic wave 24 is low on the virtual plane 22.

When an acoustic lens is used, the ultrasonic waves 24 pass through a predetermined place at the same time at a place close to the focal point of the acoustic lens. In this case, therefore, the resolution can be increased. On the other hand, at a place far from the focal point of the acoustic lens, a place where the ultrasonic waves 24 approach at the same time is away. In this case, therefore, the resolution is reduced. Then, a high-resolution place and a low-resolution place are made. In the present embodiment, since the control unit 4 moves the focal point 23 along the virtual plane 22, the ultrasonic probe 2 does not require an acoustic lens for condensing the ultrasonic waves 24 at a predetermined place. Then, the control unit 4 controls the ultrasonic element 9 so that the ultrasonic waves 24 emitted from the plurality of ultrasonic elements 9 pass through the focal point. Therefore, the ultrasonic device can suppress the occurrence of a place with low resolution. Similarly, it is also possible to suppress the occurrence of a place with low resolution in the ultrasonic probe 2.

(2) According to the present embodiment, the ultrasonic element 9 includes the piezoelectric material 31 interposed between the first electrode 30 and the second electrode 32 and the insulating layer 33 interposed between the second electrode 32 and the third electrode 34. In addition, the resistor 35 is provided so as to be connected to the second electrode 32. The control unit 4 inputs the first pulse signal 43 between the first electrode 30 and the resistor 35, and inputs the second pulse signal 44 between the first electrode 30 and the third electrode 34.

When the control unit 4 inputs the first pulse signal 43 to the resistor 35, the first pulse signal 43 is applied to the second electrode 32 through the resistor 35. Since the insulating layer 33 is interposed between the second electrode 32 and the third electrode 34, the second electrode 32, the third electrode 34, and the insulating layer 33 form the condenser 42. In addition, the control unit 4 inputs the second pulse signal 44 between the first electrode 30 and the third electrode 34.

The first pulse signal 43 and the second pulse signal 44 are signals switched between 0 V and 5 V. When the control unit 4 sets the second pulse signal 44 to 0 V and sets the first pulse signal 43 to 5 V, 5 V is applied to the condenser 42. Then, the condenser 42 holds 5 V. Then, when the control unit 4 sets the first pulse signal 43 and the second pulse signal 44 to 5 V, a voltage of 10 V that is twice the voltage of 5 V is applied to the piezoelectric element 41. In addition, since a current flows through the resistor 35, the voltage applied to the piezoelectric element 41 shifts to 5 V. Therefore, it is possible to drive the piezoelectric element 41 with a voltage of 10 V higher than 5 V output from the control unit 4. As a result, it is possible to increase the sound pressure of the ultrasonic wave 24 output from the ultrasonic element 9.

(3) According to the present embodiment, the control unit 4 drives the ultrasonic element 9 at the end of the first ultrasonic element line 12 with a voltage lower than a voltage for the ultrasonic element 9 on the central side. At this time, it is possible to reduce the amount of reflected waves from places other than the movement range of the focal point 23, compared with a case in which the control unit 4 drives the ultrasonic element 9 at the end of the first ultrasonic element line 12 with the same voltage as for the ultrasonic element 9 on the central side. Similarly, the control unit 4 drives the ultrasonic element 9 at the end of the second ultrasonic element line 13 with a voltage lower than a voltage for the ultrasonic element 9 on the central side. At this time, it is possible to reduce the amount of reflected waves from places other than the movement range of the focal point 23, compared with a case in which the control unit 4 drives the ultrasonic element 9 at the end of the second ultrasonic element line 13 with the same voltage as for the ultrasonic element 9 on the central side.

(4) According to the present embodiment, the ultrasonic element 9 of the first ultrasonic element line 12 arranged along the first direction 10 is connected through the second electrode wiring line 15. The first pulse signal 43 is input to the ultrasonic element 9 through the second electrode wiring line 15. In addition, the ultrasonic element 9 of the second ultrasonic element line 13 arranged along the second direction 11 is connected through the third electrode wiring line 16. The second pulse signal 44 is input to the ultrasonic element 9 through the third electrode wiring line 16. Therefore, it is possible to reduce the number of wiring lines compared with a case where wiring lines for supplying the first pulse signal 43 and the second pulse signal 44 are provided for each ultrasonic element 9.

### Second Embodiment

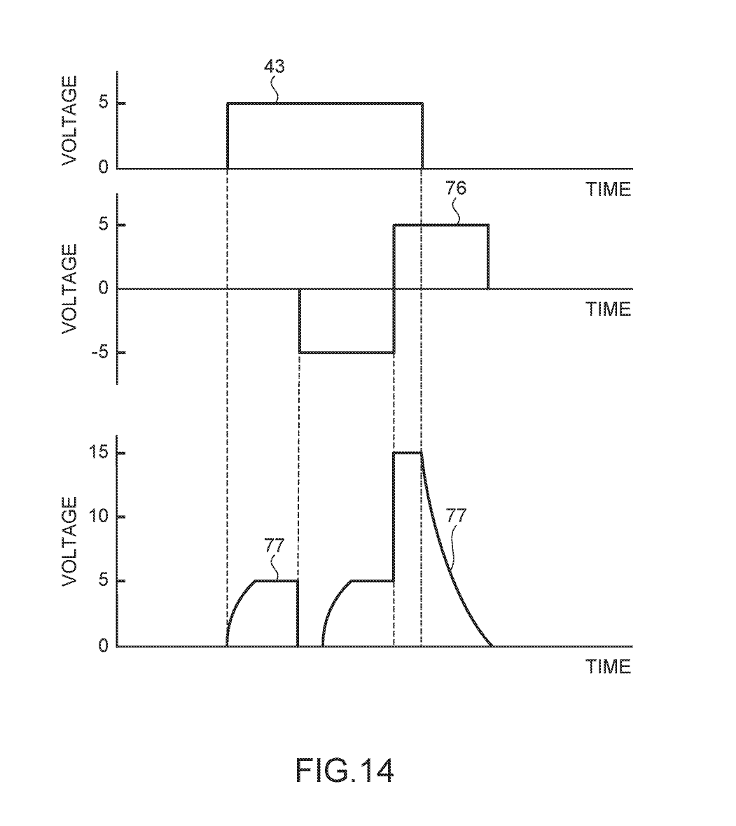

Next, an embodiment of the ultrasonic image diagnostic apparatus will be described with reference to FIGS. 13 and 14. The present embodiment is different from the first embodiment in that the shape of the second pulse signal 44 is different. In addition, the explanation of the same points as in the first embodiment will be omitted.

FIG. 13 is a circuit diagram of a piezoelectric element and a condenser. That is, in the present embodiment, as shown in FIG. 13, the second electrode wiring line 15 is connected to the other end of the resistor 35, and the first pulse signal 43 is supplied from the second electrode wiring line 15. The third electrode wiring line 16 is connected to the third electrode 34 of the condenser 42, and a second pulse signal 76 is supplied from the third electrode wiring line 16.

FIG. 14 is a time chart illustrating a driving signal. In FIG. 14, the vertical axis indicates a voltage, and the voltage on the upper side in the diagram is higher than that on the lower side. In addition, the horizontal axis indicates the transition of time, and the time transitions to the right side from the left side in the diagram. The first pulse signal 43 in the diagram is a voltage signal applied between the first electrode wiring line 21 and the second electrode wiring line 15. The second pulse signal 76 is a voltage signal applied between the first electrode wiring line 21 and the third electrode wiring line 16.

A driving waveform 77 is a waveform for driving the piezoelectric element 41, and indicates the transition of a voltage applied between the first electrode 30 and the second electrode 32. First, when the voltages of the first pulse signal 43 and the second pulse signal 76 are 0 V, the voltage of the driving waveform 77 is also 0 V.

Then, the first pulse signal 43 rises to 5 V. Then, a current flows through the resistor 35, so that electric charges are accumulated in the condenser 42. As a result, the driving waveform 77 rises to 5 V. Then, the voltage of the second pulse signal 76 drops to −5 V. Accordingly, the electric potential of the third electrode 34 becomes an electric potential of −5 V with respect to the first electrode 30. Since the condenser 42 is charged with a voltage of 5 V, the electric potential of the second electrode 32 becomes an electric potential of 0 V with respect to the first electrode 30. Since the voltage of the second electrode wiring line 15 is 5 V, a current flows through the resistor 35 so that electric charges are accumulated in the condenser 42. As a result, the driving waveform 77 rises to 5 V. Since the third electrode 34 is −5 V and the second electrode 32 is +5 V, the condenser 42 is charged with a voltage of 10 V.

Then, the second pulse signal 76 rises to 5 V. Accordingly, the electric potential of the third electrode 34 becomes an electric potential of 5V with respect to the first electrode 30. Then, since the condenser 42 is charged with a voltage of 10 V, the electric potential of the second electrode 32 becomes an electric potential of 15 V with respect to the first electrode 30.

At this time, a voltage of 15 V is applied to the piezoelectric element 41. Then, the driving waveform 77 becomes 15 V. The piezoelectric element 41 is set so as not to emit the ultrasonic wave 24 when the applied voltage is 5 V and so as to emit the ultrasonic wave 24 when the applied voltage abruptly rises from 5 V to 10 V. Therefore, when the second pulse signal 76 becomes 5 V, the piezoelectric element 41 emits the ultrasonic wave 24.

Since the electric potential of the second electrode wiring line 15 is 5 V, the charge of the second electrode 32 moves to the second electrode wiring line 15 through the resistor 35. In addition, since the first pulse signal 43 becomes 0 V, the charge of the second electrode 32 further moves to the second electrode wiring line 15 through the resistor 35. Accordingly, the voltage of the driving waveform 77 gradually drops to become 0 V. Then, the voltage of the second pulse signal 76 also drops to 0 V. Then, the piezoelectric material 31 returns to the initial state in which no voltage is applied. Therefore, the control unit 4 can drive the piezoelectric element 41 with a voltage of 15 V using each signal of the first pulse signal 43 having a voltage of +5 V and the second pulse signal 76 having a voltage of ±5 V.

The piezoelectric element 41 having a higher driving voltage can make the sound pressure of the ultrasonic wave 24 higher. Therefore, it is possible to transmit the ultrasonic wave 24 far away in the case of driving the piezoelectric element 41 with a voltage of 15 V compared with a case of driving the piezoelectric element 41 with a voltage of 10 V. Accordingly, it is possible to capture an ultrasonic image of a deep place of the living body 19.

The invention is not limited to the embodiments described above, and various modifications or improvements can be made within the technical idea of the invention by those skilled in the art. Modification examples will be described below.

### MODIFICATION EXAMPLE 1

In the first embodiment described above, the virtual plane 22 is set at the central position of the first ultrasonic element line 12 in the first direction 10. However, the position of the virtual plane 22 may be a place other than the center of the first ultrasonic element line 12. Alternatively, the position of the first ultrasonic element line 12 in the first direction 10 may be changed by the operator. In this case, it is possible to easily change the imaging place.

The entire disclosure of Japanese Patent Application No. 2016-038646 filed on Mar. 1, 2016 is expressly incorporated by reference herein.

What is claimed is:

1. An ultrasonic device, comprising:
an ultrasonic element group in which first ultrasonic element lines are arranged along a second direction crossing a first direction, a plurality of ultrasonic elements being arranged along the first direction in each of the first ultrasonic element lines; and
a control unit that controls the ultrasonic element group,
wherein the control unit moves a focal point, which is a place through which ultrasonic waves emitted from the plurality of ultrasonic elements pass, along a virtual plane.

2. The ultrasonic device according to claim 1,
wherein each of the ultrasonic elements includes a piezoelectric material interposed between first and second electrodes and an insulating layer interposed between the second electrode and a third electrode,
an electrical resistor is provided in the second electrode, and
the control unit inputs a first pulse signal between the first electrode and the electrical resistor, and inputs a second pulse signal between the first and third electrodes.

3. The ultrasonic device according to claim 1,
wherein the control unit drives the ultrasonic element at an end of the first ultrasonic element line with a voltage lower than a voltage for the ultrasonic element on a central side.

4. The ultrasonic device according to claim 2, further comprising:
a first wiring line that is connected to each ultrasonic element of the first ultrasonic element line to transmit the first pulse signal; and
a second wiring line that is connected to the ultrasonic element of a second ultrasonic element line, which is arranged along the second direction, to transmit the second pulse signal.

5. An ultrasonic probe, comprising:
an ultrasonic element group in which first ultrasonic element lines are arranged along a second direction crossing a first direction, a plurality of ultrasonic elements being arranged along the first direction in each of the first ultrasonic element lines; and
a control unit that controls the ultrasonic element group,
wherein the control unit moves a focal point, which is a place through which ultrasonic waves emitted from the plurality of ultrasonic elements pass, along a virtual plane.
